(19) United States
(12) Patent Application Publication
(10) Pub. No.: US 2025/0339159 A1
(43) Pub. Date: Nov. 6, 2025

(54) ENHANCED CATHETERS AND METHODS OF USE

(71) Applicant: Uri STEIN, Ramat Gan (IL)

(72) Inventor: Uri STEIN, Ramat Gan (IL)

(21) Appl. No.: 18/860,749

(22) PCT Filed: Apr. 28, 2023
(86) PCT No.: PCT/IB2023/054432

§ 371 Date: Oct. 28, 2024

Related U.S. Application Data
(60) Provisional application No. 63/337,084, filed on Apr. 30, 2022.
(60) Provisional application No. 63/483,292, filed on Feb. 5, 2023.

(51) Int. Cl. A61B 17/22; A61B 17/00; A61M 25/01

(52) U.S. Cl. CPC A61B 17/22; A61M 25/0141; A61M 25/0147; A61B 2017/00154; A61B 2017/00862; A61B 2017/22094; A61M 2205/0216; A61M 2205/33; A61M 2210/12

(57) ABSTRACT

A system and method for steering a catheter and removing a blockage from a vessel, wherein removing the blockage includes providing a blockage removal system including a steerable catheter and a controller, wherein the controller is configured to steer the catheter and cause the catheter to move with a hammering movement; inserting the catheter into the vessel; and activating using the controller of a hammering action of the catheter against the blockage to thereby remove the blockage.

### FIELD

The present disclosure is of enhanced catheters for removing a blockage from a tubular vessel of a patient and for use in minimally invasive surgery.

### BACKGROUND

Numerous devices may be used for the removal or opening of a blockage in a blood vessel. Such a blockage may be caused by, for example, atherosclerosis or thrombosis. Currently available devices may use rotating cutting tips, lasers, or tips with heating elements to remove or open blockages of vessels. Some devices may use ultrasonic pressure at the blockage site.

Many of these current approaches may result in inadvertent cutting of the wall of the blood vessel while being used to remove a blockage, with resultant ill effects. Further, current approaches may be too aggressive, failing to offer subtle control of the device thus potentially damaging the blood vessel by putting too much pressure on the blockage during the attempted removal. Yet further, some approaches, such as ultrasonic pressure devices may not work for removing blockages having soft material and cutting type devices may not work for blockages having calcified material.

Thus, there is a need for an alternative solution for removal or opening of a blockage in a blood vessel that offers fine control and reduces the risk to patients.

Steerable catheters are increasingly used for minimally invasive surgery (MIS) where the catheter may be inserted into the vascular system, gastrointestinal tract, or airway for both diagnosis and treatment. Hu (Hu, Xiaohua, et al. “Steerable catheters for minimally invasive surgery: a review and future directions.” Computer Assisted Surgery 23.1 (2018): 21-41) describes “tendon-steered” steerable catheters as being relatively bulky, limiting the miniaturization potential of such catheters. Further, the friction between the tendon and guide channel as well as the backlash in the joints of the tendon may make such steerable catheters difficult to control.

Thus, there is a need for a steerable catheter that does not require multiple tendons or wires for operation and that is easier and more predictable to control.

### SUMMARY

The present disclosure is directed to systems and methods for removal or opening of a blockage in a vessel. In some embodiments, the described system includes a catheter that may be introduced into the blocked vessel, the catheter configured to perform a repetitive hammering action against the blockage in order to remove or open the blockage.

In some embodiments, the catheter may be made from an elastic material and the hammering action may be provided by repeatedly causing the elastic catheter to be compressed and then expanded against the blockage to thus hammer against the blockage. In some embodiments, the hammering action may be controlled by a controller that may be operated by a human operator such as a medical professional while viewing the position of the catheter inside a blood vessel using medical imaging techniques as known in the art.

In some embodiments, the controller may provide control over the hammering rate and force of the hammering action while the catheter is moved by the operator to make physical contact with the blockage until the blockage has been removed.

The present disclosure is further directed to steerable catheters with a distal end that may be partially formed from elastic materials of varying elastic moduli such that compression of the catheter may result in a bending of the distal end for steering of the catheter. The compression of the distal end may be performed by pulling on a single pulling wire positioned within the catheter. Advantageously, use of a single pulling wire does not impose size constraints such as in typical 3 or 4-wire catheters and the steerable catheter disclosed herein may thus be provided with a smaller diameter. In some embodiments, the bending/steering may be controlled by a controller that may be operated by a human operator such as a medical professional while viewing the position of the catheter inside a vessel using medical imaging techniques as known in the art.

In some embodiments, the steerable catheter as disclosed herein may be configured to provide a hammering effect such that the catheter may be steered to the site of a blockage in a vessel and activated to perform a repetitive hammering action against the blockage in order to remove or open the blockage.

As used herein “distal” refers to those parts of the disclosed catheters that are furthest from the controller and “proximal” refers to parts that are closest to the controller. The term “removal” of a blockage may refer to either opening or removal of some or all of the blockage material. The term “blockage” as used herein may refer to any form of vessel blockage or partial vessel blockage that requires removal.

The term “vessel” as used herein may refer to any tubular tissue within a body (human or animal) such as but not limited to veins, arteries, ducts, intestines, or other tubular structures. The term “operator” as used herein may refer to any medical professional that may the use the device and method disclosed herein for removing a blockage in a vessel.

In some embodiments, a system for removing a blockage from a vessel includes: a catheter; and a pulling wire attached to an inside distal end of the catheter; wherein pulling on the pulling wire towards a proximal side of the catheter causes the catheter to compress and wherein subsequent releasing of the pulling wire causes the catheter to un-compress for generating a hammering action at a distal tip of the catheter. In some embodiments, the catheter includes a portion formed from an elastic material.

In some embodiments, the uncompressing of the catheter imparts a force at the catheter tip of between 10 N and 1000 N. In some embodiments, the system further includes a controller including a movement generator configured to pull on the pulling wire and to subsequently release the pulling wire. In some embodiments, the movement generator imparts a continual hammering rate of up to 100 cycles/sec.

In some embodiments, the catheter has a compressed length that is between 1 mm and 10 mm shorter than the full uncompressed length of the catheter. In some embodiments, the catheter is dimensioned for insertion into a vessel. In some embodiments, the catheter has an external diameter of between 0.5 to 10 mm. In some embodiments, the catheter has one or more radiopaque portions.

In some embodiments, the movement generator includes at least one disc with a protruding rod configured to pull and then subsequently release an engagement bar on a proximal end of the pulling wire, and wherein a proximal edge of the catheter presses against and/or may be fixedly attached to an outer surface of the movement generator.

In some embodiments, a method for removing a blockage from a vessel includes: providing a blockage removal system including a catheter and a controller, wherein the controller is configured to cause the catheter to move with a hammering movement; inserting the catheter into the vessel; and activating using the controller of a hammering action of the catheter against the blockage to thereby remove the blockage.

In some embodiments, the catheter includes a portion formed from an elastic material. In some embodiments, the uncompressing of the catheter imparts a force at the catheter tip of between 10 N and 1000 N. In some embodiments, the blockage removal system further includes a controller including a movement generator configured to pull on the pulling wire and to subsequently release the pulling wire. In some embodiments, the movement generator imparts a continual hammering rate of up to 100 cycles/sec.

In some embodiments, the catheter has a compressed length that is between 1 mm and 10 mm shorter than the full uncompressed length of the catheter. In some embodiments, the catheter is dimensioned for insertion into a vessel. In some embodiments, the catheter has an external diameter of between 0.5 to 10 mm. In some embodiments, the catheter has one or more radiopaque portions.

In some embodiments, the movement generator includes at least one disc with a protruding rod configured to pull and then subsequently release an engagement bar on a proximal end of the pulling wire, and wherein a proximal edge of the catheter presses against and/or may be fixedly attached to an outer surface of the movement generator.

In some embodiments, a non-transitory computer readable medium contains instructions that when executed by at least one processor, cause the at least one processor to perform operations for generating a hammering motion in a catheter, the operations including: instructing a movement generator to pull on a pulling wire and to subsequently release the pulling wire, wherein the pulling wire is attached to an inside distal end of the catheter, and wherein the catheter is at least partially formed of an elastic material.

In some embodiments, a steerable catheter includes: a catheter including a distal end includes elastic materials each having different elastic moduli arranged along substantially opposite sides of the distal end; and a single pulling wire attached to an inside distal end of the catheter; wherein pulling on the single pulling wire towards a proximal side of the catheter causes the catheter to compress and bend in a direction of a side of the distal end having the elastic material having a lower elastic modulus to thereby steer the catheter in the direction of the bend.

In some embodiments, the elastic material with the higher elastic modulus has an elastic modulus that is 2-100 times larger than the elastic modulus of the elastic material having the lower elastic modulus. In some embodiments, a bending radius of the distal end of the catheter is up to 180 degrees. In some embodiments, the system further includes a controller configured to pull on the pulling wire according to a desired bend radius.

In some embodiments, the catheter is dimensioned for insertion into a vessel. In some embodiments, the catheter has an external diameter of between 0.5 to 10 mm. In some embodiments, the catheter has one or more radiopaque portions. In some embodiments, the distal end is between 10-100 mm long. In some embodiments, the distal end is between 1%-100% of the catheter length.

In some embodiments, a method of steering a catheter through a vessel includes: providing the steerable catheter as described above; inserting the catheter into the vessel; when reaching a bend in the vessel, pulling on the pulling wire to cause the steering catheter to bend while guiding the steering catheter through the bend in the vessel.

In some embodiments, a non-transitory computer readable medium may contain instructions that when executed by at least one processor, cause the at least one processor to perform operations for steering a steering catheter as described above, the operations including: instructing the controller to manipulate the pulling wire according to a desired bend radius of the distal tip.

In some embodiments, a system for removing a blockage from a vessel includes: a catheter including a distal end comprising elastic materials each having different elastic moduli arranged along substantially opposite sides of the distal end; and a single pulling wire attached to an inside distal end of the catheter; wherein pulling on the single pulling wire towards a proximal side of the catheter causes the catheter to compress and bend in a bend direction of a side of the distal end having the elastic material having a lower elastic modulus to thereby steer the catheter in the direction of the bend, wherein pulling on the pulling wire towards a proximal side of the catheter causes the catheter to compress and wherein subsequent releasing of the pulling wire causes the catheter to un-compress for generating a hammering action at a distal tip of the catheter.

It is to be understood that the figures and descriptions of the present invention have been simplified to illustrate elements that are relevant for a clear understanding of the present invention, while eliminating, for the purpose of clarity, many other elements found in typical surgical apparatuses, systems, and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to the disclosed elements and methods known to those skilled in the art.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Aspects, embodiments, and features disclosed herein will become apparent from the following detailed description when considered in conjunction with the accompanying drawings. In the drawings:

FIG. 1A is a schematic illustration of a system 100 for removal of a blockage from a vessel according to some embodiments.

FIGS. 1B-1D are illustrations showing operating principles of a system for removal of a blockage from a vessel according to some embodiments.

FIGS. 1E-1H show an exemplary device for manipulating the movement of part of a system for removal of a blockage from a vessel according to some embodiments.

FIG. 2A is a flow diagram showing a process for use of a system for removal of a blockage from a vessel according to some embodiments.

FIGS. 2B-2E are illustrative drawings showing a process for use of a system for removal of a blockage from a vessel according to some embodiments.

FIG. 3A is a schematic illustration of a steerable catheter system according to some embodiments.

FIGS. 3B-3D are illustrations showing operating principles of a steerable catheter system according to some embodiments.

FIG. 3E is a schematic illustration of a steerable catheter system configured for removal of a blockage from a vessel according to some embodiments.

FIG. 4A is a flow diagram showing a process for use of a steerable catheter system according to some embodiments.

FIGS. 4B-4E are illustrative drawings showing a process for use of a steerable catheter system according to some embodiments.

### DETAILED DESCRIPTION

In the detailed description, numerous specific details are set forth in order to provide a thorough understanding of the disclosure. However, it will be understood by those skilled in the art that these are specific embodiments and that the present disclosure may be practiced also in different ways that embody the characterizing features of the disclosure as described and claimed herein. In the drawings and descriptions set forth, identical reference numerals indicate those components that are common to different embodiments or configurations. The present disclosure will be more fully understood from the following detailed description of embodiments thereof, taken together with the drawings.

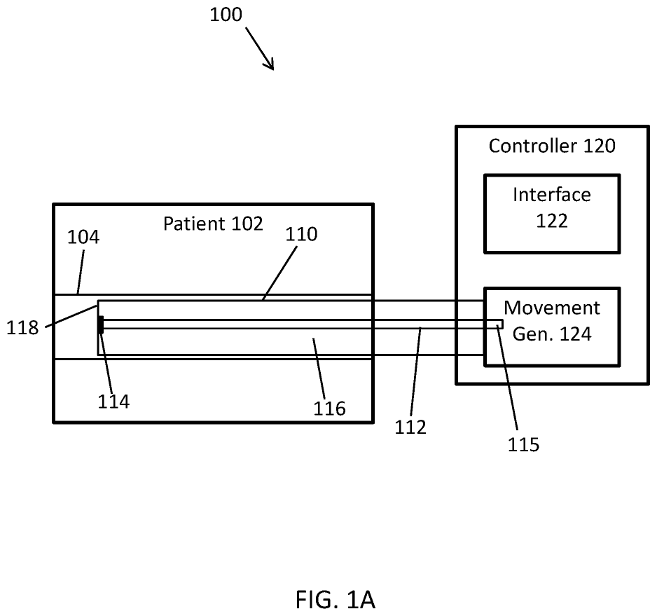

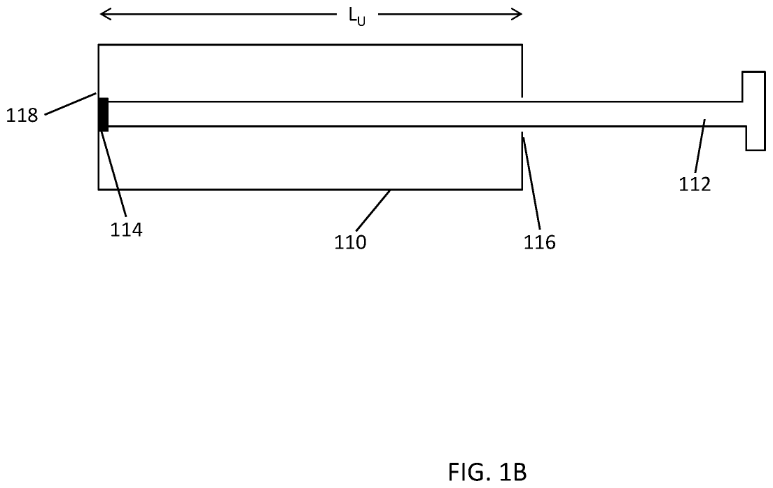

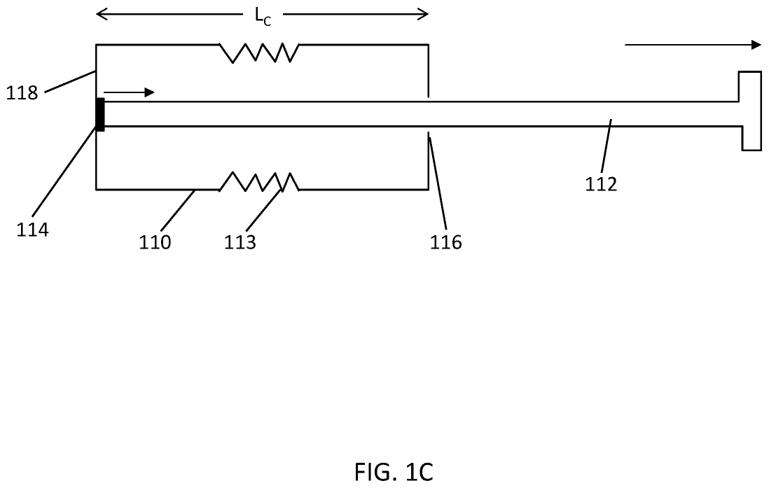

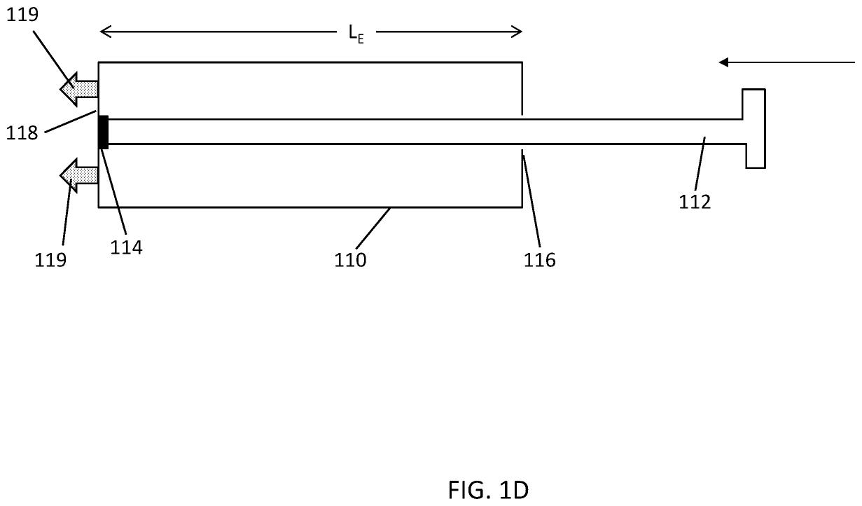

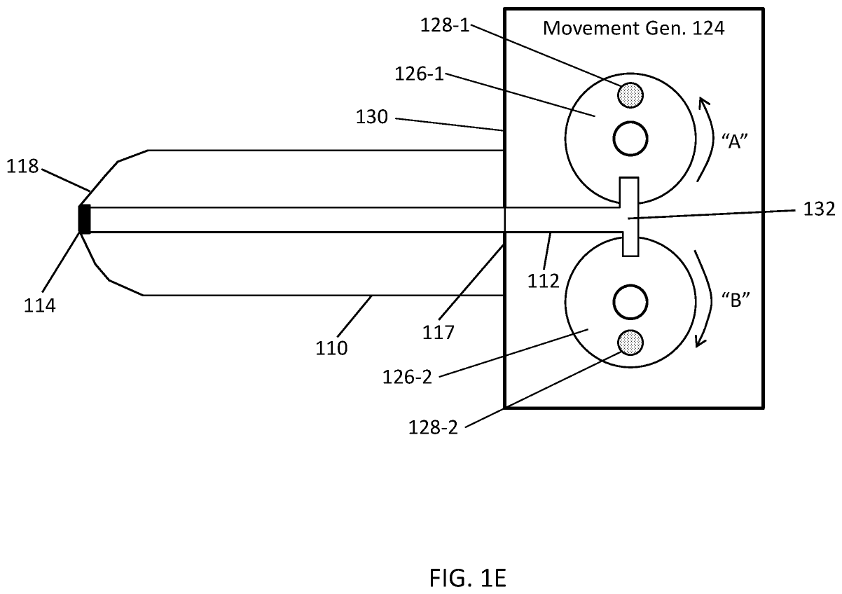

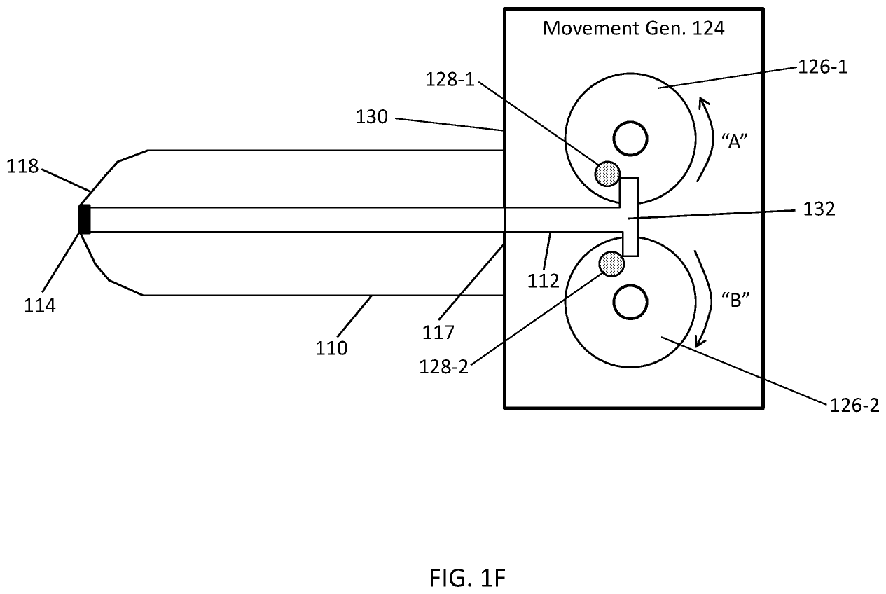

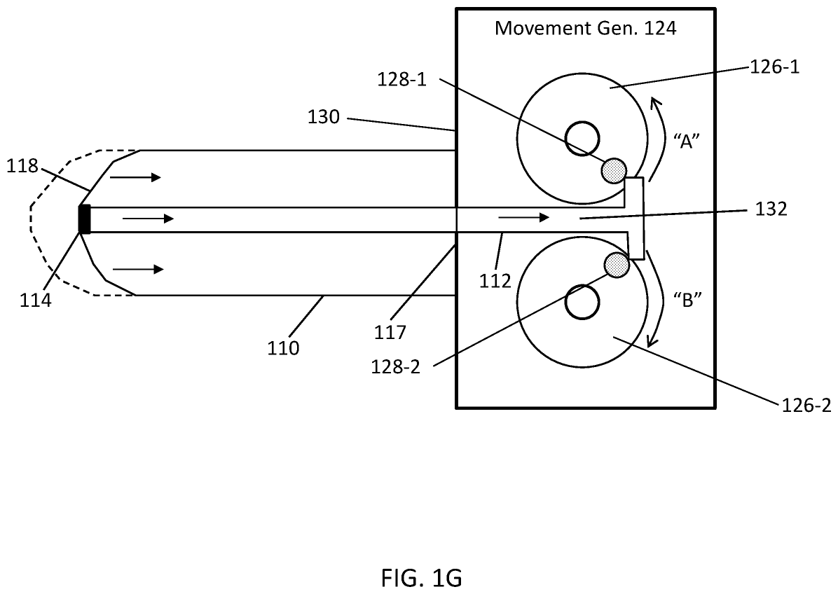

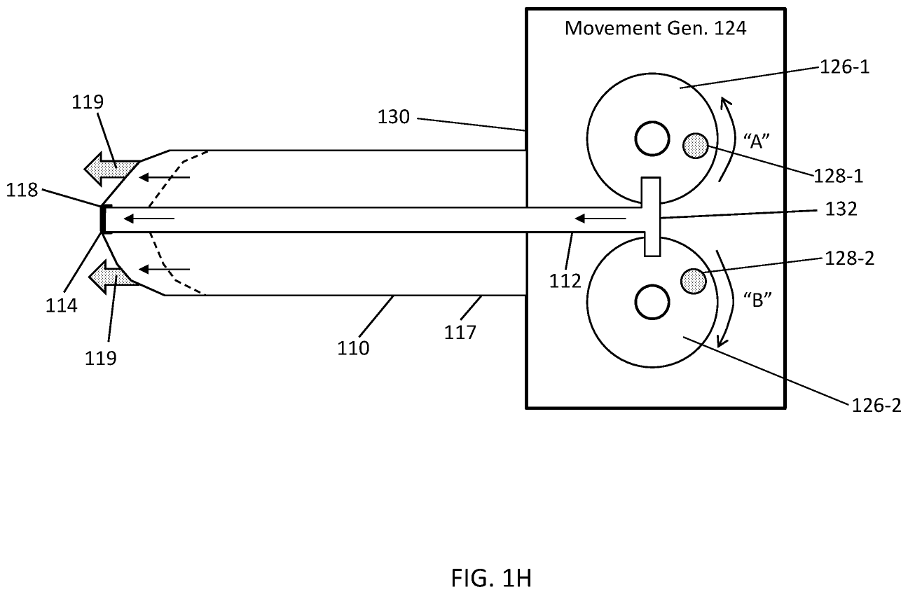

The present disclosure describes technological improvements for devices and methods for removal of a blockage in a vessel and for steerable catheters, the devices and methods offering fine control and reducing the risk to patients. FIG. 1A is a schematic illustration of a system 100 for removal of a blockage from a vessel. FIGS. 1B-1D are illustrations showing operating principles of a system for removal of a blockage from a vessel. FIGS. 1E-1H show an exemplary device for manipulating the movement of part of a system for removal of a blockage from a vessel.

As shown in FIGS. 1A-1H, system 100 may include a catheter 110, pulling wire 112, and controller 120. Catheter 110 may be inserted into vessel 104 of a patient 102 for removal of a blockage in vessel 104. Pulling wire 112 may be fixedly attached to catheter 110 at an inner distal connection point 114 of catheter 110 and pass through aperture 116 on the proximal edge 117 of catheter 110. Pulling wire 112 may engage at a proximal end 115 with movement generator 124.

Catheter 110 may be configured for insertion into vessel 104 of patient 102. Catheter 110 may thus have an external diameter suitable for fitting within the inner diameter of vessel 104 and allowing movement of catheter 110 within vessel 104. In some embodiments, the outer diameter of catheter 110 may be between 0.5 mm to 10 mm. Pulling wire 112 may fit within catheter 110 and in some embodiments may have an external diameter of between 0.1 mm to 1 mm.

Catheter 110 may include a portion formed from an elastic material enabling catheter 110 to be compressed along its length as further described below. In some embodiments, catheter 100 may have a compressed length L_{C} (FIG. 1C) that is between 1 mm and 10 mm shorter than the full uncompressed length L_{U} (FIGS. 1B, 1D) of catheter 110. In some embodiments, tip 118 of catheter 110 may have a conical/pointed shape such as shown in FIGS. 1E-1H. In some embodiments, tip 118 of catheter 110 may have barbs or similar protrusions (not shown).

In some embodiments, catheter 110 may be fully or partially formed from a biocompatible material including but not limited to plastic, steel, titanium, elastomers, or nitinol. In some embodiments, catheter 110 may have one or more radiopaque portions to enable viewing of these radiopaque portions by a medical imaging device (not shown) when catheter 110 is inserted into a vessel 104 of a patient 102 to thereby determine the position of catheter 110 inside patient 102.

Controller 120 is a computing device including at least one processor. Controller 120 may include a non-transitory computer readable medium containing instructions that when executed by the at least one processor are configured to perform the functions and/or operations necessary to provide the functionality described herein.

Controller 120 may include a controller user interface 122 that provides interface components as known in the art such as but not limited to a screen, keyboard, mouse, or other components for use by an operator (not shown) such as a medical professional to control the operation of catheter 110 and to receive information about the status of catheter 110.

Controller 120 may include movement generator 124 (FIGS. 1E-1H) connected to pulling wire and configured for pulling and releasing pulling wire 112 which may be attached thereto to thereby cause a hammering action in catheter 110. Movement generator 124 may generate a range of movement hammering rates (impacts per period of time) and amplitudes (length of compression generated in catheter 110 by pulling on wire 112).

In some embodiments, catheter 110 may be disposable such that it is used once for each patient. In some embodiments, where catheter 110 is disposable, pulling wire 112 may connect to controller 120 via a connector (not shown) for one-time attachment of catheter 110 to controller 120.

FIG. 1B shows catheter 110 in a fully uncompressed state. As shown in FIG. 1C when pulling wire 112 is pulled in a proximal direction by controller 120, catheter tip 118 is also pulled in a proximal direction such that catheter 110 becomes compressed. FIG. 1C illustrates a compressed region 113 but this is merely illustrative and the compression of catheter 110 may take place without any visible deforming of the body of catheter 110. As show in FIG. 1D, pulling wire 112 is released by controller 120 and catheter 110 rapidly expands (springs) back into an uncompressed state. The forward movement of catheter tip 118 as part of the uncompressing may create a force indicated by arrows 119 that is imparted to blockages in contact with catheter tip 118 to create a hammering effect against the blockage.

A non-limiting example of a movement generator 124 including operation thereof is shown in FIGS. 1E-1H. It should be appreciated that other embodiments of movement generator 124 are contemplated and the embodiment shown in FIGS. 1E-1H is exemplary. In some embodiments, movement generator 124 may include discs 126-1 and 126-2 that are rotated by one or more motors (not shown) such as in directions “A” and “B”. Protruding rods 128-1 and 128-2 may be fixedly attached to discs 126-1 and 126-2 respectively and may rotate as discs 126-1 and 126-2 rotate. Proximal edge 117 of catheter 110 may press against and/or may be fixedly attached to an outer surface 130 of movement generator 124 to thereby provide counter pressure to the pulling of pulling wire 112 to thereby cause compression of catheter 110. Pulling wire 112 may be formed into a T-shaped engagement bar 132 on a proximal end 115 thereof.

FIG. 1E shows catheter 110 in a fully uncompressed state. As shown in FIG. 1F, as discs 126-1 and 126-2 rotate, protruding rods 128-1 and 128-2 rotate until making contact with engagement bar 132. As shown in FIG. 1G, protruding rods 128-1 and 128-2 may pull pulling wire 112 in a proximal direction, to thereby pull attachment point 114 and catheter tip 118 in a proximal direction, while proximal edge 117 of catheter 110 presses against outer surface 130 of movement generator 124, such that catheter 110 becomes compressed. As show in FIG. 1H, as discs 126-1 and 126-2 continue to rotate, protruding rods 128-1 and 128-2 rotate until they no longer make contact with engagement bar 132 thus releasing pulling wire 112 such that catheter 110 rapidly expands (springs) back into an uncompressed state. The forward movement of catheter tip 118 as part of the uncompressing may create a force indicated by arrows 119 that is imparted to blockages in contact with catheter tip 118 to create a hammering effect against the blockage. In some embodiments, the hammering effect may be in a direction substantially perpendicular to distal end 118 of catheter 110, i.e., substantially in the direction of movement of catheter 110 through vessel 104.

In some embodiments, the force thus imparted by catheter tip 118 may be between 10 N and 1000 N. In some embodiments, controller 120 (using movement generator 124) may repeat the pulling/releasing actions on pulling wire 112 to thereby impart a continual hammering effect. In some embodiments, controller 120 may be configured to impart a hammering rate of up to 1 cycles/sec. In some embodiments, controller may be configured to impart a hammering rate of between 1 to 100 cycles/sec. In some embodiments, the hammering rate may be continual or may be variable over a time period. In some embodiments, controller 120 may pull pulling wire 112 by an increased distance to thereby increase the compression of catheter 110 and to thereby increase the subsequent force imparted by catheter tip 118 upon release of pulling wire 112. Controller 120 may thus control the force of impact (hammering) and/or the hammering rate.

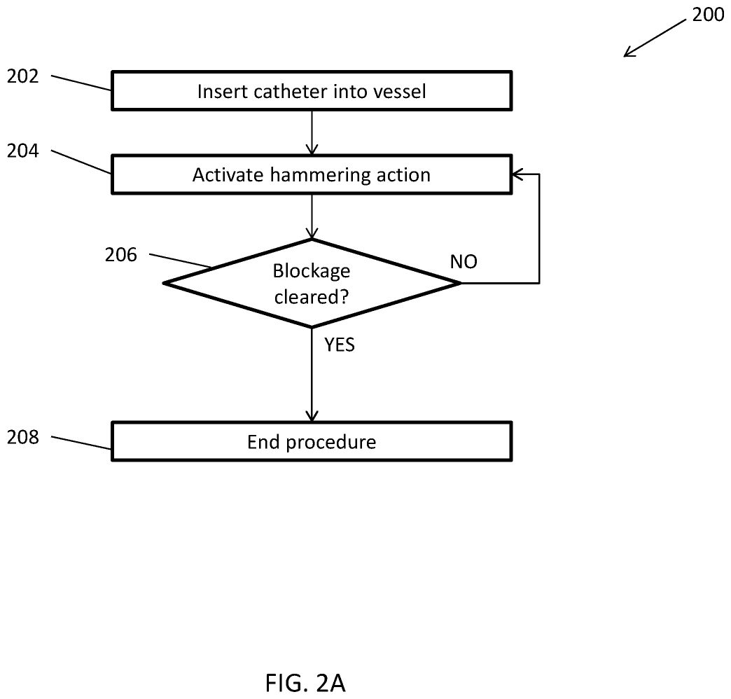

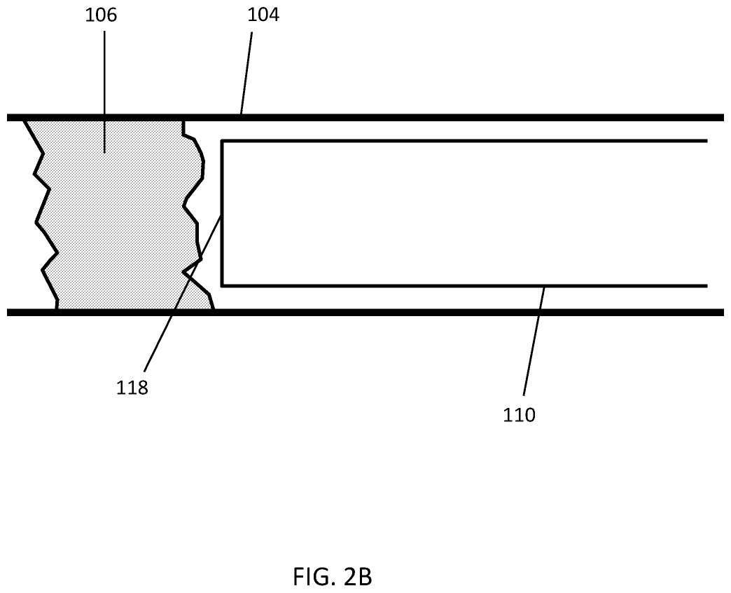

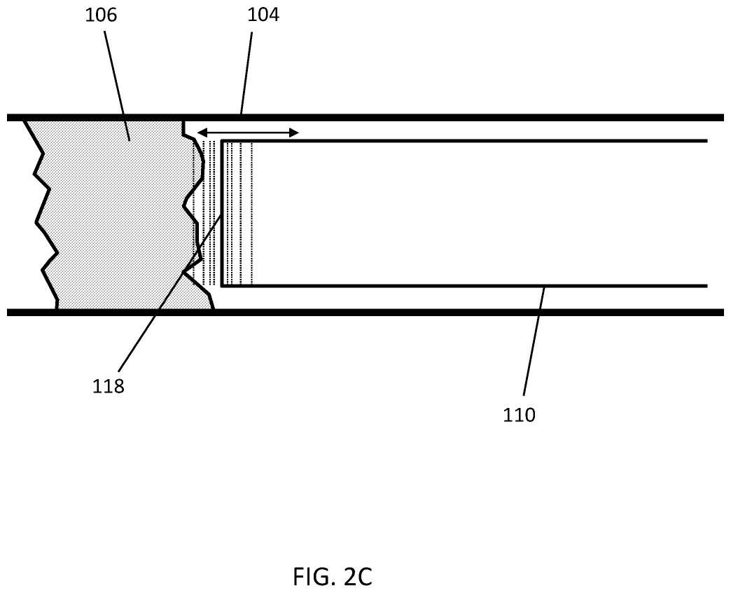

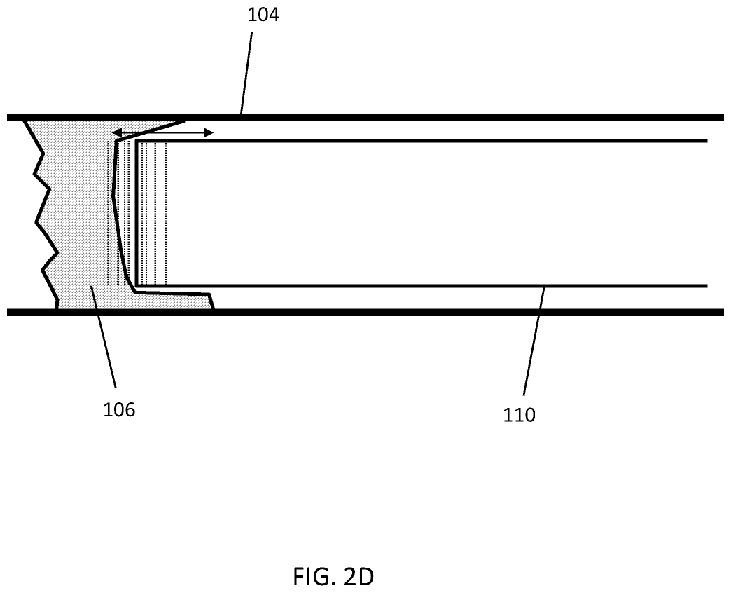

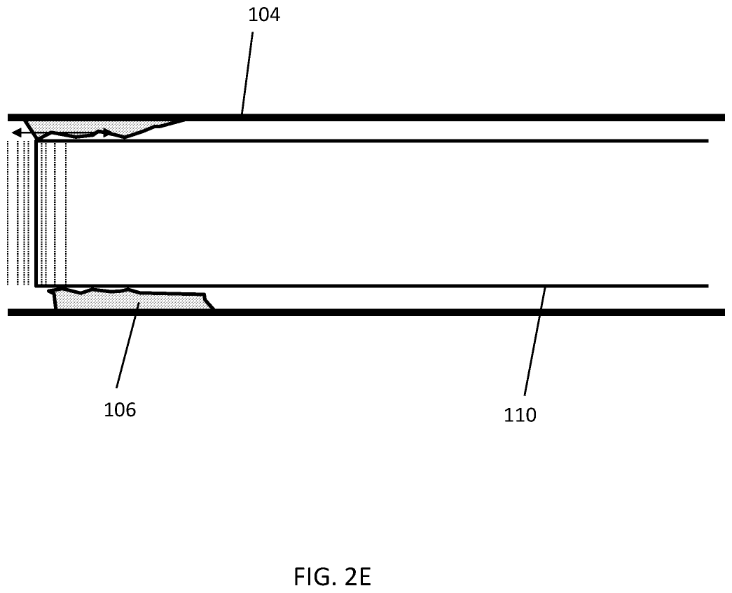

FIG. 2A is a flow diagram showing a process 200 for removal of a blockage from a vessel according to some embodiments. FIGS. 2B-2E are illustrative drawings showing a process 200 for use of a system for removal of a blockage from a vessel according to some embodiments. A non-transitory computer readable medium may contain instructions that when executed by at least one processor perform some or all of the operations described at each step as part of process 200. The non-transitory computer readable medium and at least one processor may correspond to controller 120. Process 200 may be performed by controller 120 or alternatively by controller 120 along with a human operator.

In step 202 of process 200 as shown in FIG. 2B catheter 110 may be inserted into vessel 104 containing a blockage 106. Catheter 110 may be moved along vessel 104 while being viewed externally to the patient using a medical imaging device until catheter 110 reaches blockage 106 (FIG. 2B) and catheter tip 118 appears to be substantially in contact with blockage 106. Movement of catheter 110 may be performed by an operator feeding (pushing) catheter 110 into vessel 104.

In step 204, once catheter tip 118 is in contact with blockage 106, a hammering action of catheter tip 118 (FIG. 2C) against blockage 106 may be initiated such as by interaction with controller 120. In some embodiments, the rate of hammering and/or the force (amplitude) of hammering of catheter tip 118 on blockage 106 may be adjusted by an operator such as by interaction with controller 120.

In step 206, an operator may assess, such as by using medical imaging, whether blockage 106 has been removed. If it is determined that blockage 106 is still substantially in place, then step 204 may be repeated including adjustment of the rate and/or force of hammering. As shown in FIG. 2D, the hammering action may continue as catheter 110 removes blockage 106. If, in step 206, it is determined that blockage 106 has been removed (such as shown in FIG. 2E), then in step 208 the procedure may be terminated and catheter 110 may be withdrawn from vessel 104 and patient 102.

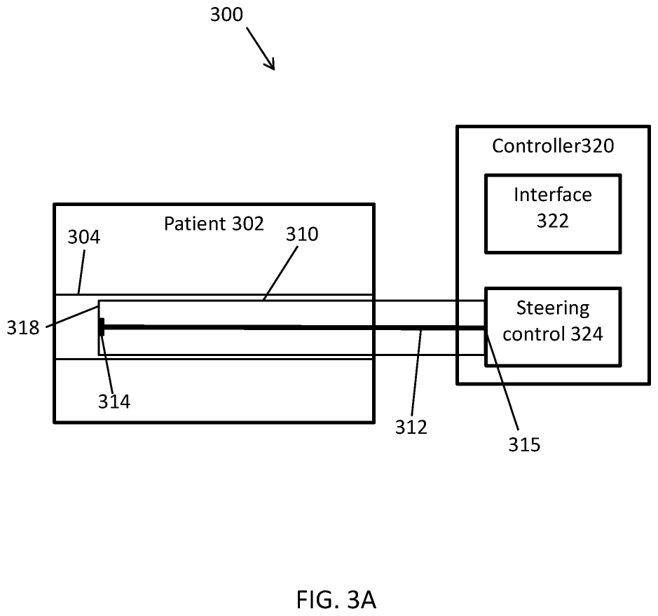

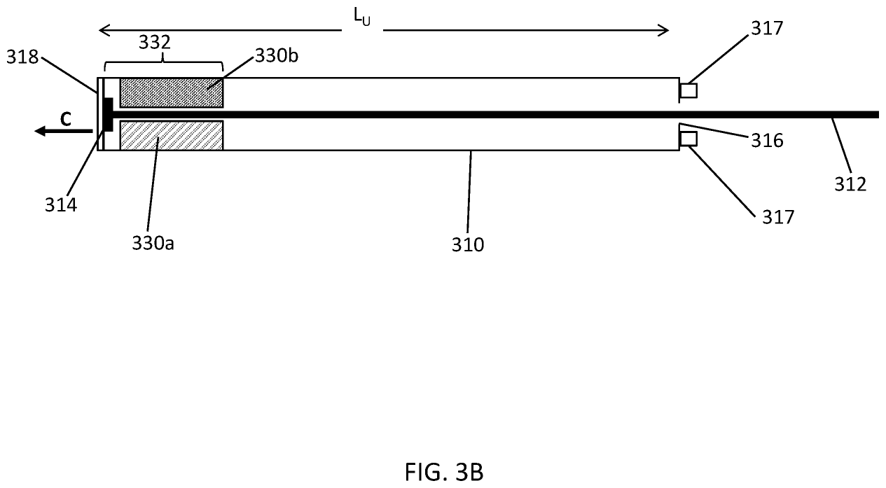

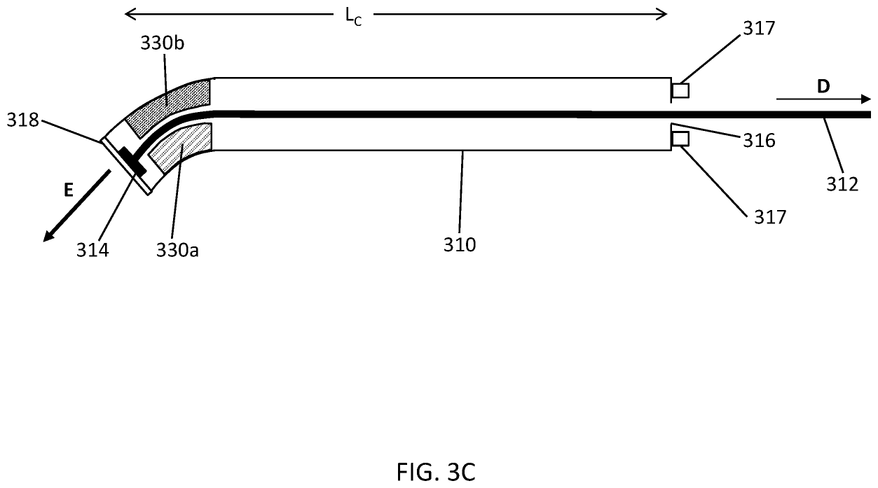

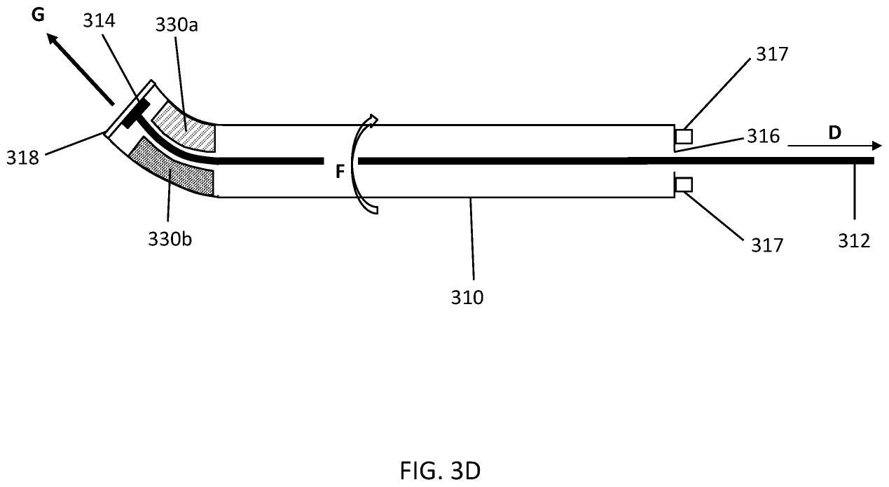

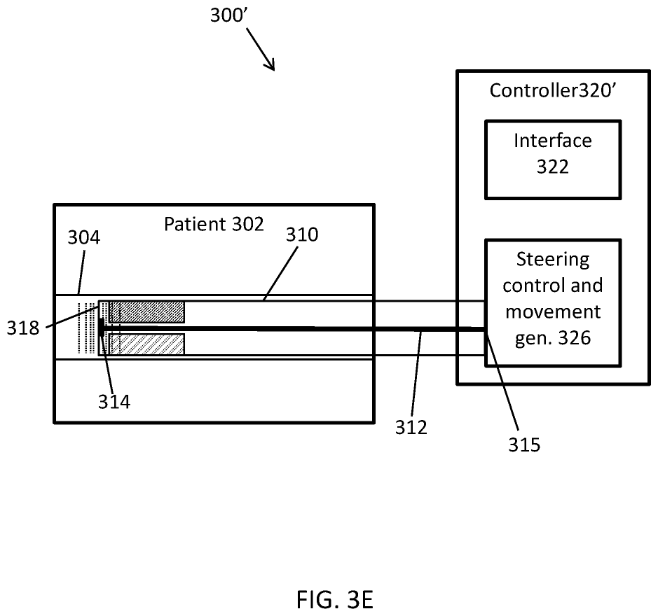

The present disclosure further describes technological improvements for steerable catheters and methods of use of these steerable catheters. FIG. 3A is a schematic illustration of a steerable catheter system 300. FIGS. 3B-3D are illustrations showing operating principles of steerable catheter system 300.

As shown in FIGS. 3A-3D, system 300 may include a catheter 310, pulling wire 312, and controller 320. Catheter 310 may be inserted into vessel 304 of a patient 302 for performing MIS or for other medical procedures (such as removing or opening a blockage) or examinations. Pulling wire 312 may be fixedly attached to catheter 310 at an inner distal connection point 314 of catheter 310 and pass through aperture 316 on the proximal edge 317 of catheter 310. Pulling wire 312 may engage at a proximal end 315 with a steering controller 324 and catheter 310 may abut a counterpressure surface 317 that may be positioned within or outside of controller 320.

Catheter 310 may be configured for insertion into vessel 304 of a patient 302. Catheter 310 may thus have an external diameter suitable for fitting within the inner diameter of vessel 304 and allowing movement of catheter 310 within vessel 304. In some embodiments, the outer diameter of catheter 310 may be between 0.5 mm to 10 mm. Pulling wire 312 may fit within catheter 310 and in some embodiments may have an external diameter of between 0.1 mm to 1 mm. In some embodiments, pulling wire 312 may be formed from a metal.

Catheter 310 may include a portion formed from an elastic material enabling catheter 310 to be compressed along its length as further described below. In some embodiments, catheter 310 may include multiple distal portions 330 of elastic material along a distal portion 332 of catheter 310 where each of portions 330 may have a different elastic modulus. For example, portions 330a and 330b each have different elastic modulus-illustrated in FIGS. 3B-3D as blocks with differing patterns where portion 330a has a lower elastic modulus than portion 330b. In some embodiments, portions 330 are of the same size. In some embodiments, portions 330 may be positioned substantially directly opposite one another around a circumference of catheter 310. In some embodiments, multiple portions 330 may be distributed around a circumference of catheter 310 where at least two of the multiple portions 330 have different elastic moduli from one another or where all of the portions 330 have different elastic moduli from one another or where a portion of the portions 330 have different elastic moduli from a different portion of the portions 330. In some embodiments, multiple portions 330 may be equally angularly distributed around a circumference of catheter 310.

FIG. 3B shows catheter 310 in a fully uncompressed state. In an uncompressed state catheter 310 may be moved through a vessel 304 in a straight direction as shown by arrow “C”. As shown in FIG. 3C, when pulling wire 312 is pulled in a proximal direction (shown by arrow “D”) by controller 320, catheter tip 318 is also pulled in a proximal direction while counterpressure surfaces 317 prevent a proximal end of catheter 310 from being pulled in a proximal direction such that catheter 310 becomes compressed. The differing elastic moduli of portions 330a and 330b result in different levels of compression of portions 330a and 330b resulting in catheter 310 bending towards the portion having the lower elastic modulus (here shown as portion 330a). Although two portions 330a and 330b are shown, it should be appreciated that multiple portions of varying elastic modulus may be provided around the distal end of catheter 310. In some embodiments, distal end 332 including portions 330 may be between 10-100 mm in length as measured from tip 318. The elastic modulus of each of portions 330 may be selected to affect the amount that distal portion 332 is bent.

When moved within vessel 304, catheter 310 will thus be steered and move in a direction indicated by arrow “D” when compressed and bent. In some embodiments, catheter 310 may be rotated such as indicated by arrow “F” (FIG. 3D) before pulling wire 312 is pulled in order to bend catheter 310 such that when moved within a vessel 304, catheter 310 may thus be steered and move in a direction indicated by arrow “G” when compressed and bent. Releasing pulling wire 312 may return catheter 310 to an unbent state (FIG. 3B) for steering and moving in a straight direction “C” through vessel 304.

It should be appreciated that the degree of bending will depend on the elastic moduli of portions 330 as well as on the amount that pulling wire 312 is pulled, i.e.: to steer catheter 310 around a curve in vessel 304, pulling wire 312 may be pulled to bend catheter 310 according to the degree of turning required. It should be appreciated that the combination of bending of catheter 310 (arrows “E” and “G”) and rotating of catheter 310 (arrow “F”) may steer catheter 310 with any required bending angle through vessel 304.

In some embodiments, catheter 310 may have a compressed length L_{C} (FIG. 3C) that is between 1 mm and 10 mm shorter than the full uncompressed length L_{U} (FIG. 3B) of catheter 310. In some embodiments, the elastic material 330 with the higher elastic modulus 330b has an elastic modulus that is 2-100 times larger than the elastic modulus of the elastic material having the lower elastic modulus 330a. In some embodiments, a bending radius of the distal end of catheter 310 is up to 180 degrees. In some embodiments, a distal end is between 1%-100% of the length of catheter 310.

In some embodiments, catheter 310 may be fully or partially formed from a biocompatible material including but not limited to plastic, steel, titanium, elastomers, or nitinol. In some embodiments, catheter 310 may have one or more radiopaque portions to enable viewing of these radiopaque portions by a medical imaging device (not shown) when catheter 310 is inserted into a vessel 304 of a patient 302 to thereby determine the position of catheter 310 inside patient 302.

Controller 320 is a computing device including at least one processor. Controller 320 may include a non-transitory computer readable medium containing instructions that when executed by the at least one processor are configured to perform the functions and/or operations necessary to provide the functionality described herein.

Controller 320 may include a controller user interface 322 that provides interface components as known in the art such as but not limited to a screen, keyboard, mouse, or other components for use by an operator (not shown) such as a medical professional to control the operation of catheter 310 and to receive information about the status of catheter 310.

Controller 320 may include steering controller 324 connected to pulling wire for manipulating (pulling, releasing, and moving backwards and forwards) pulling wire 312 for bending catheter 310 to thereby steer catheter 310 through vessel 304 and for moving catheter 310 forwards and backwards along vessel 304. In some embodiments, steering controller 324 may rotate catheter 310 (as shown by arrow F in FIG. 3D) to align catheter 310 before pulling on pulling wire 312 to bend and thereby steer catheter 310 through vessel 304. In some embodiments, steering controller 324 may pull pulling wire 312 by an increased amount to thereby increase the compression of catheter 310 and to thereby increase the subsequent bending amount to match a bend in a vessel being navigated.

In some embodiments, catheter 310 may be disposable such that it is used once for each patient. In some embodiments, where catheter 310 is disposable, pulling wire 312 may connect to controller 320 via a connector (not shown) for one-time attachment of catheter 310 to controller 320.

FIG. 3E shows a schematic illustration of a steerable catheter system 300′ configured for removal of a blockage from a vessel according to some embodiments. In some embodiments, system 300′ is the same as system 300 but makes use of controller 320′. Controller 320′ is the same as controller 320 but includes a combined steering control and movement generator 326 in place of steering controller 324. Pulling wire 312 may be connected to steering control and movement generator 326.

Steering control and movement generator 326 may be configured for manipulating (pulling and releasing) pulling wire 312 which may be attached thereto to thereby cause a hammering action in catheter 310. Steering control and movement generator 326 may generate a range of movement hammering rates (impacts per period of time) and amplitudes (length of compression generated in catheter 310) by pulling on wire 312. Steering control and movement generator 326 may have the configuration shown in FIGS. 1E-1H. In some embodiments, the force thus imparted by catheter 310 may be between 10 N and 1000 N. In some embodiments, steering control and movement generator 326 may repeat the pulling/releasing actions on pulling wire 312 to thereby impart a continual hammering effect. In some embodiments, a hammering rate of up to 1 cycles/sec may be provided. In some embodiments, a hammering rate of between 1 to 100 cycles/sec may be provided. In some embodiments, the hammering rate may be continual or may be variable over a time period. In some embodiments, steering control and movement generator 326 may pull pulling wire 312 by an increased distance to thereby increase the compression of catheter 310 and to thereby increase the subsequent force imparted by catheter tip 318 upon release of pulling wire 312. Steering control and movement generator 326 may thus control the force of impact (hammering) and/or the hammering rate.

Steering control and movement generator 326 may be configured for pulling and releasing pulling wire 312 for bending catheter 310 to thereby steer catheter 310 through a vessel 304. In some embodiments, steering control and movement generator 326 may rotate catheter 310 (as shown by arrow F in FIG. 3D) to align catheter 310 before pulling on pulling wire 312 to bend and thereby steer catheter 310 through vessel 304. In some embodiments, steering control and movement generator 326 may pull pulling wire 312 by an increased amount to thereby increase the compression of catheter 310 and to thereby increase the subsequent bending amount to match a bend in a vessel being navigated. In some embodiments, steering control and movement generator 326 may be used to hammer at a blockage while being steered towards parts of the blockage.

In some embodiments, where catheter 310 is disposable, pulling wire 312 may connect to controller 320′ via a connector (not shown) for one-time attachment of catheter 310 to controller 320′. When used for clearing of blockages, catheter tip 318 of catheter 310 may have a conical/pointed shape or may have barbs or similar protrusions.

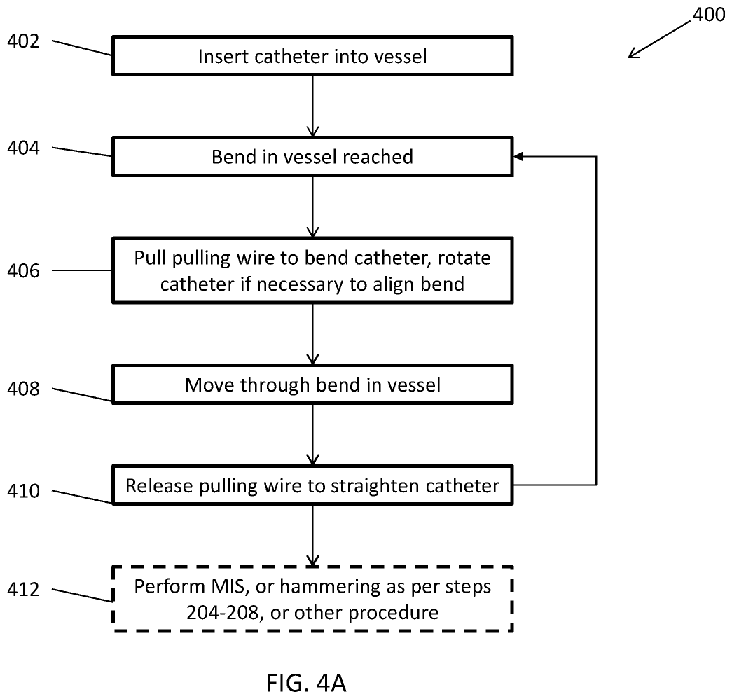

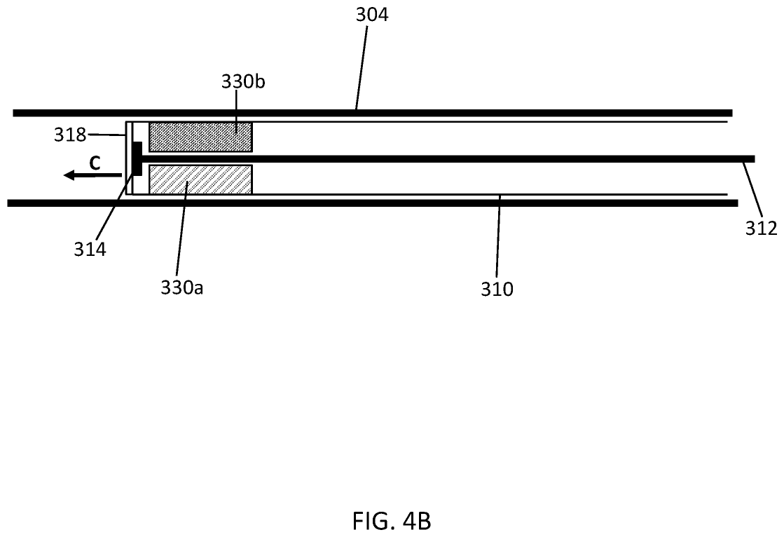

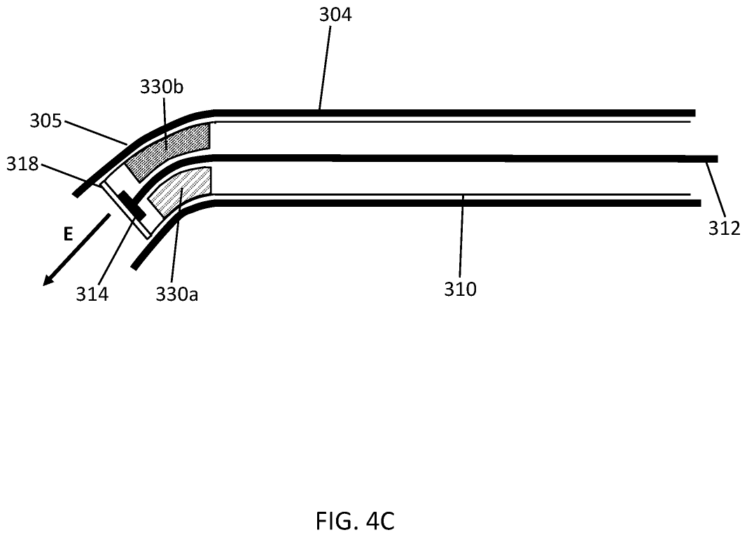

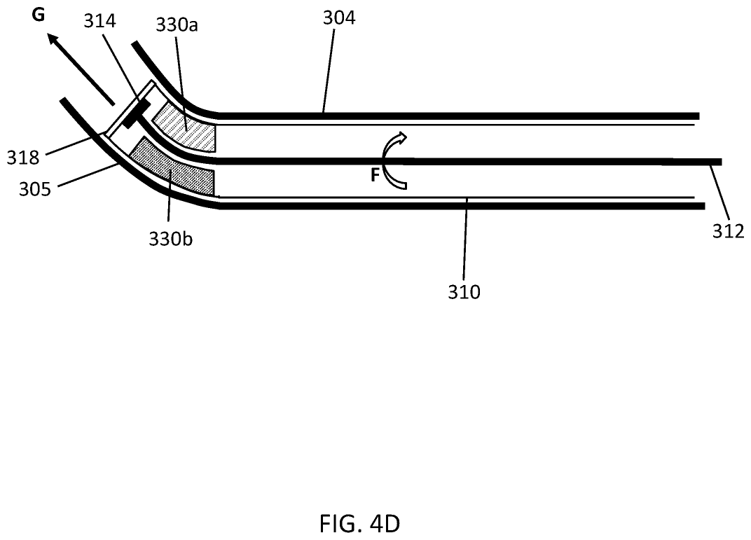

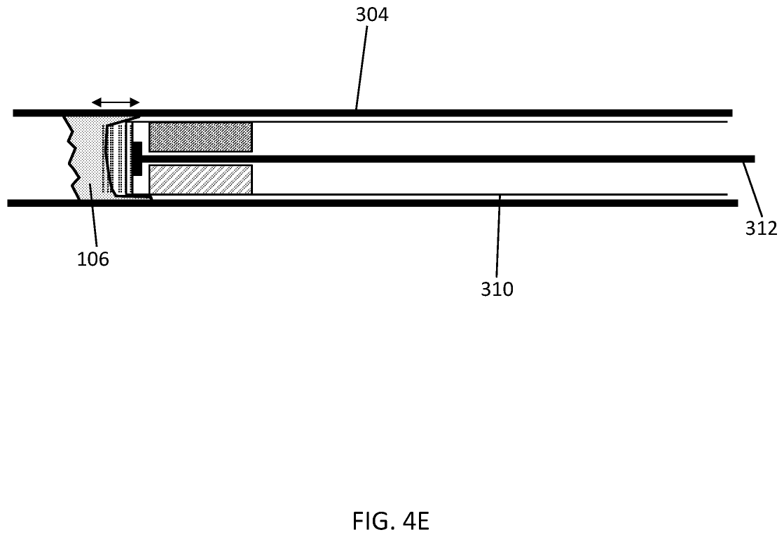

FIG. 4A is a flow diagram showing a process 400 for operation of a steering catheter according to some implementations. FIGS. 4B-4E are illustrative drawings showing a process 400 for use of a steerable catheter according to some implementations. A non-transitory computer readable medium may contain instructions that when executed by at least one processor perform some or all of the operations described at each step as part of process 400. The non-transitory computer readable medium and at least one processor may correspond to controller 320. Process 400 may be performed by controller 320 or alternatively by controller 320 along with a human operator.

In step 402 of process 400 as shown in FIG. 4B catheter 310 may be inserted into vessel 304. Catheter 310 may be moved along vessel 304 (FIG. 4B) while being viewed externally to the patient using a medical imaging device until, in step 404, catheter 310 reaches a bend 305 in vessel 304. In some embodiments, forward and backward movement of catheter 310 may be performed by an operator or by controller 320 feeding (pushing) catheter 310 into vessel 304.

In step 406, a bending of catheter 310 in order to steer through a bend in vessel 304 (FIGS. 3C and 3D) may be initiated such as by interaction with controller 320 that may pull on pulling wire 312. In some embodiments, the angle of bending of catheter 310 may be adjusted by an operator such as by interaction with controller 320. Alternatively, a human operator may pull on pulling wire 312 to thereby bend catheter 310. In some embodiments, catheter 310 may be rotated to adjust the alignment of catheter 310 such that the bending direction of catheter 310 (being a function of the different elastic moduli of portions 330) may be aligned with the bend in vessel 304. Rotating of catheter 310 may be performed by controller 320 or alternatively by a human operator.

In step 408, catheter 310 may be moved through the bend in vessel 304 (FIGS. 3C and 3D). In some embodiments, steps 406 and 408 are performed concurrently for steering of catheter 310, thus, catheter 310 may be bent and/or rotated while being moved through a bend in vessel 304.

In step 410, having completed the navigation of catheter 310 through vessel 304, pulling wire 312 may be released so as to straighten catheter 310. The release of pulling wire 312 may be performed by controller 320 and/or by an operator.

Steps 404 to 410 may then be repeated as necessary until catheter 310 reaches a target area for step 412 where MIS or another medical procedure or examination may be performed.

In some embodiments, in step 412, where the aim is removal or opening of a blockage 106, and blockage 106 has been reached steps 204-208 of process 200 may be performed using system 300′ (FIG. 3E) where controller 320′ includes combined steering control and movement generator 326. It should be appreciated that the small contractions required for producing a hammering effect by tip 318 may result in bending of distal end 332 of catheter 310 where the bending effect may be utilized in directing the hammering against the blockage in order to more thoroughly clear the blockage.

Unless otherwise defined, all technical and scientific terms used herein have the same meaning as commonly understood by one of ordinary skill in the art to which this disclosure belongs. The materials, methods, and examples provided herein are illustrative only and not intended to be limiting.

Implementation of the method and system of the present disclosure involves performing or completing certain selected tasks or steps manually, automatically, or a combination thereof. Moreover, according to actual instrumentation and equipment of preferred embodiments of the method and system of the present disclosure, several selected steps could be implemented by hardware or by software on any operating system of any firmware or a combination thereof. For example, as hardware, selected steps of the disclosure could be implemented as a chip or a circuit. As software, selected steps of the disclosure could be implemented as a plurality of software instructions being executed by a computer using any suitable operating system. In any case, selected steps of the method and system of the disclosure could be described as being performed by a data processor, such as a computing platform for executing a plurality of instructions.

Although the present disclosure is described with regard to a “computing device”, a “computer”, or “device”, or “mobile device” on a “computer network” or simply “network”, it should be noted that optionally any device featuring a data processor and the ability to execute one or more instructions may be described as a computer or one of the interchangeable terms listed above, including but not limited to any type of personal computer (PC), a server, a cellular telephone, an IP telephone, a smartphone, or a PDA (personal digital assistant). Any two or more of such devices in communication with each other may optionally form a “network”.

It is appreciated that certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

It is to be understood that the disclosure is not limited in its application to the details set forth in the description contained herein or illustrated in the drawings. The disclosure is capable of other embodiments and of being practiced and carried out in various ways. Those skilled in the art will readily appreciate that various modifications and changes can be applied to the embodiments of the disclosure as hereinbefore described without departing from its scope, defined in and by the appended claims.

1. A system for removing a blockage from a vessel comprising:
a catheter; and
a pulling wire attached to an inside distal end of the catheter;
wherein pulling on the pulling wire towards a proximal side of the catheter causes the catheter to compress and wherein subsequent releasing of the pulling wire causes the catheter to un-compress for generating a hammering action at a distal tip of the catheter.

2. The system of claim 1, wherein the catheter includes a portion formed from an elastic material.

3. The system of claim 1, wherein the uncompressing of the catheter imparts a force at the catheter tip of between 10 N and 1000 N.

4. The system of claim 1, further comprising a controller including a movement generator configured to pull on the pulling wire and to subsequently release the pulling wire.

5. The system of claim 4, wherein the movement generator imparts a continual hammering rate of up to 100 cycles/sec.

6. The system of claim 1, wherein the catheter has a compressed length that is between 1 mm and 10 mm shorter than the full uncompressed length of the catheter.

7. The system of claim 1, wherein the catheter is dimensioned for insertion into a blood vessel.

8. The system of claim 1, wherein the catheter has an external diameter of between 0.5 to 10 mm.

9. (canceled)

10. The system of claim 4, wherein the movement generator includes at least one disc with a protruding rod configured to pull and then subsequently release an engagement bar on a proximal end of the pulling wire, and wherein a proximal edge of the catheter presses against and/or may be fixedly attached to an outer surface of the movement generator.

11. A method for removing a blockage from a vessel comprising:
providing a blockage removal system including a catheter and a controller, wherein the controller is configured to cause the catheter to move with a hammering movement;
inserting the catheter into the vessel; and
activating using the controller of a hammering action of the catheter against the blockage to thereby remove the blockage.

12-13. (canceled)

14. The method of claim 11, wherein the blockage removal system further comprises a controller including a movement generator configured to pull on the pulling wire and to subsequently release the pulling wire.

15-21. (canceled)

22. A steerable catheter comprising:
a catheter including a distal end comprising elastic materials each having different elastic moduli arranged along substantially opposite sides of the distal end; and
a single pulling wire attached to an inside distal end of the catheter;
wherein pulling on the single pulling wire towards a proximal side of the catheter causes the catheter to compress and bend in a direction of a side of the distal end having the elastic material having a lower elastic modulus to thereby steer the catheter in the direction of the bend.

23. The system of claim 22, wherein the elastic material with the higher elastic modulus has an elastic modulus that is 2-100 times larger than the elastic modulus of the elastic material having the lower elastic modulus.

24. The system of claim 22, wherein a bending radius of the distal end of the catheter is up to 180 degrees.

25. The system of claim 22, further comprising a controller configured to pull on the pulling wire according to a desired bend radius.

26. The system of claim 22, wherein the catheter is dimensioned for insertion into a vessel.

27. The system of claim 22, wherein the catheter has an external diameter of between 0.5 to 10 mm.

28. (canceled)

29. The system of claim 22, wherein the distal end is between 10-100 mm long.

30. The system of claim 22, wherein the distal end is between 1%-100% of a catheter length.

31. A method of steering a catheter through a vessel comprising:
providing the steerable catheter of claim 22;
inserting the catheter into a vessel;
when reaching a bend in the vessel, pulling on the pulling wire to cause the steering catheter to bend while guiding the steering catheter through the bend in the vessel.

32-33. (canceled)
